(19) United States
(12) Patent Application Publication
(10) Pub. No.: US 2020/0178877 A1
(43) Pub. Date: Jun. 11, 2020

(54) ANIMAL MODEL FOR GENERATING A STEREOTYPE OF FOCAL ONSET SEIZURES

(71) Applicants: CHUNG SHAN MEDICAL UNIVERSITY HOSPITAL, Taichung City (TW); CHUNG SHAN MEDICAL UNIVERSITY, Taichung City (TW)

(72) Inventors: YUE-LOONG HSIN, Taichung City (TW); SYU-JYUN PENG, Zhubei City (TW); CHENG-SIU CHANG, Taichung City (TW); FU-YUAN SHIH, Kaohsiung City (TW)

(21) Appl. No.: 16/283,736

(22) Filed: Feb. 22, 2019

(30) Foreign Application Priority Data
Dec. 11, 2018 (TW) 107144615

(51) Int. Cl. A61B 5/00; A61K 49/00; A61B 5/0484; A61B 5/04; A61D 7/04

(52) U.S. Cl. CPC A61B 5/4094; A61K 49/0008; A61B 5/0484; A61B 2503/42; A61B 5/6868; A61D 7/04; A61B 2503/40; A61B 5/04001

(57) ABSTRACT

It includes the steps of (1) preparing step; (2) anesthetizing step; (3) electrodes installing step; and (4) focal onset seizures inducing step. First, a live animal is anesthetized. The electrodes are installed on a working zone of its brain. A first chemical liquid is injected beneath the cerebral cortex of this animal. At two different times, a first electric stimulation and a second electric stimulation are activated by the controller respectively so as to generate a first and a second focal onset seizure accordingly. Thus, it can generate focal onset seizures. It is easy to change the electric stimulation position, numbers and type. In addition, the survival rate of a larger-sized animal after anesthesia is higher.

### BACKGROUND OF THE INVENTION

### 1. Field of the Invention

The present invention is related to an animal model for generating a stereotype of focal onset seizures. In this invention, it can generate focal onset seizures. It is easy to change the electric stimulation position, range, times and type. In addition, the survival rate of a larger-sized animal after anesthesia is higher.

### 2. The Related Arts

The happening time of epilepsy (or focal onset seizures) is unpredictable, so the research for epilepsy related fields becomes extremely difficult. In the past, a laboratory mouse is used for animal model test or experiment. However, the volume of the brain of a mouse is too small when it compares with the volume of the brain of a human. So, it is not suitable. In recent years, some search institute uses monkey for animal model test or experiment. Although the volume of the brain of a monkey is larger than the volume of the brain of a mouse, but it still smaller than the volume of the brain of a human. Thus, it is not an ideal animal to be tested.

With regard to the related prior arts, U.S. Pat. No. 9,629,346 is an animal model for epilepsy and method for producing the same. It uses the mouse as the animal to be tested. However, it mainly discusses the gene sequence rather than discusses about how to generate epilepsy.

U.S. Pat. No. 6,484,059 relates a method for optimized brain stimulation for treating movement disorders. It utilizes an electrode which is deeply inserted inside a specific position of a brain for stimulation. It is not to study about how to generate epilepsy.

Besides, there is no prior arts using a larger-sized animal as the testing animal in an animal model test or experiment.

### SUMMARY OF THE INVENTION

An object of the present invention is to provide an animal model for generating a stereotype of focal onset seizures. Advantages of the present invention include that it can generate focal onset seizures; it is easy to change the electric stimulation position, range, times and type; and the survival rate of a larger-sized animal after anesthesia is higher. In particular, conventional technology did not use a larger-sized animal as the testing object. No conventional technology studied how to generate epilepsy. Now, it is can be solved by the present invention.

Technical solutions to solve the above-mentioned drawbacks are to provide an animal model for generating a stereotype of focal onset seizures comprising the steps of:

(1) preparing step;

(2) anesthetizing step;

(3) electrodes installing step; and

(4) focal onset seizures inducing step.

The above objects and advantages of the present invention can be easily understood in depth from the following detailed descriptions of preferred embodiments of the present invention and accompanying drawings.

The present invention is further illustrated and explained in details by the following preferred embodiments of the present invention and accompanying drawings as follows.

### BRIEF DESCRIPTION OF THE DRAWINGS

FIG. 1 is a flow chart of the present invention.

FIG. 2 is a perspective view illustrating the device of this invention.

FIG. 3 is a view showing the process one of the animal model test in this invention.

FIG. 4 is a view showing the process two of the animal model test in this invention.

FIG. 5 is a view showing the process three of the animal model test in this invention.

FIG. 6 is a view showing the process four of the animal model test in this invention.

FIG. 7 is a view showing the process five of the animal model test in this invention.

FIG. 8 is a view showing the process six of the animal model test in this invention.

FIG. 9 illustrates the electrodes of the present invention.

FIG. 10 is another view showing the electrodes of the present invention.

FIG. 11 is a view depicting the arrangement of the electrodes in this invention.

FIG. 12 is a view showing the brainwave signals recorded by the electrodes when the first focal onset seizure is generated.

FIG. 13 is a view showing the brainwave signals recorded by the electrodes when the second focal onset seizure is generated.

### DETAILED DESCRIPTION OF PREFERRED EMBODIMENT

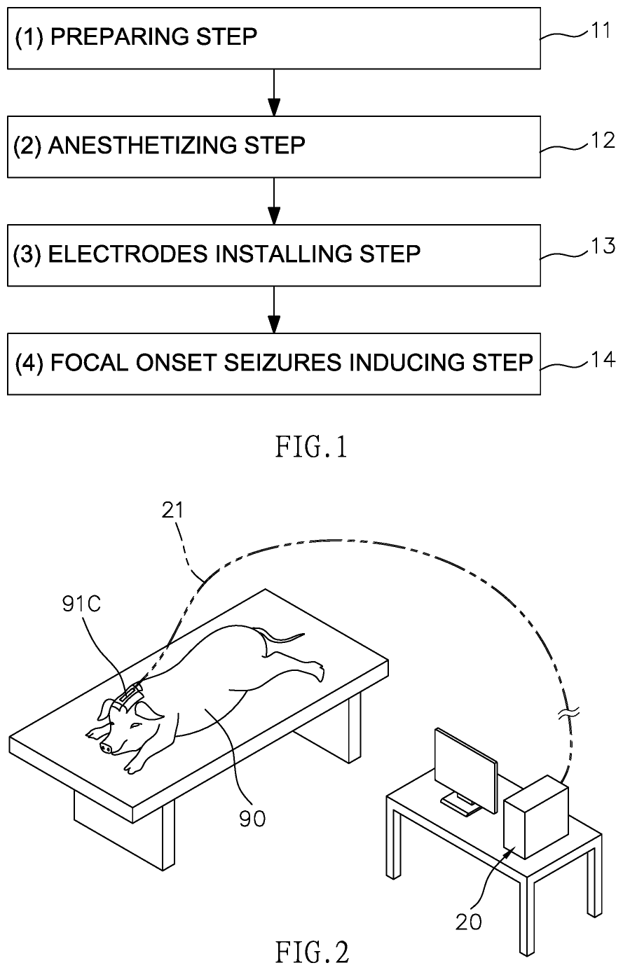

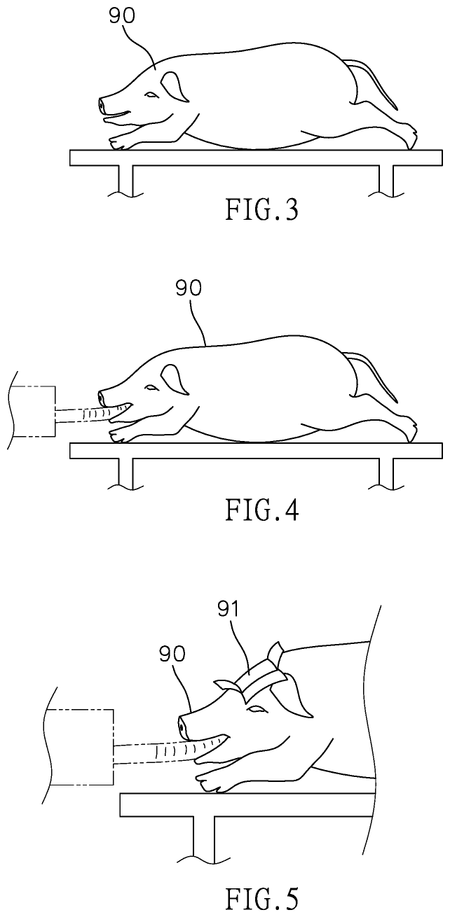

Referring to FIGS. 1, 2, and 3, the present invention is related to an animal model for generating a stereotype of focal onset seizures. It mainly comprises the steps of (1) preparing step 11, (2) anesthetizing step 12, (3) electrodes installing step 13, and (4) focal onset seizures inducing step 14.

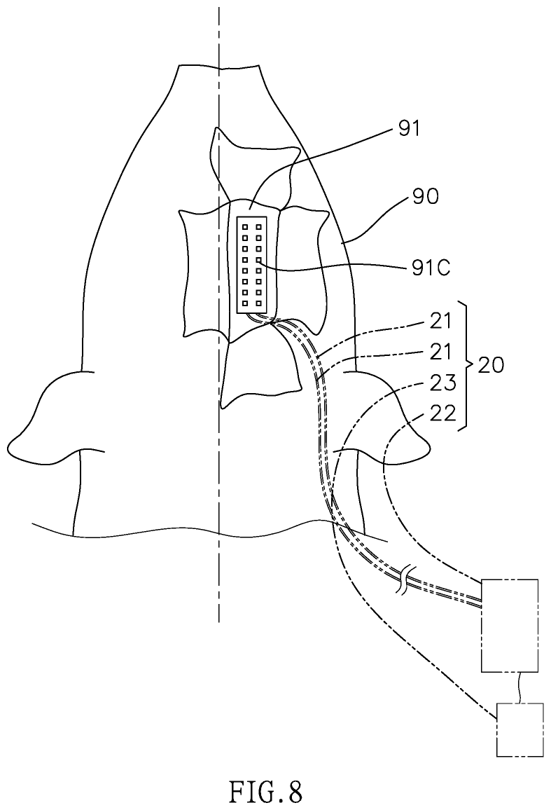

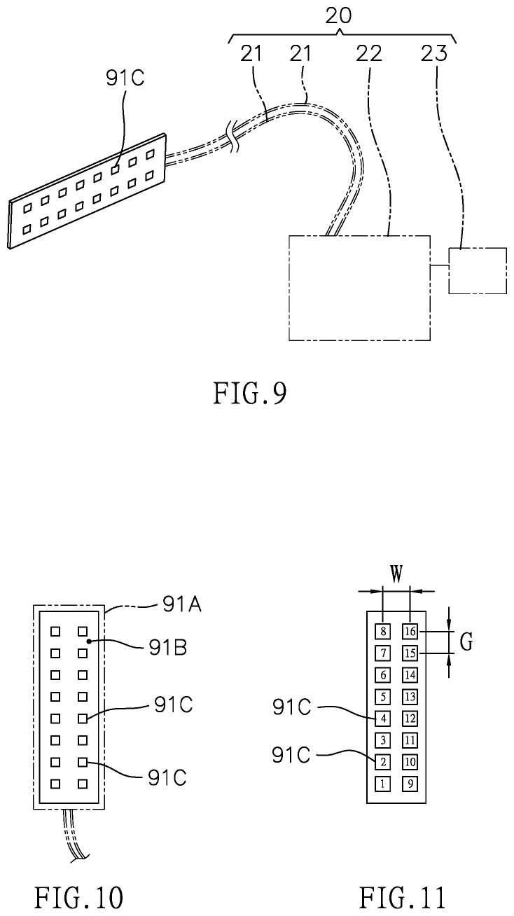

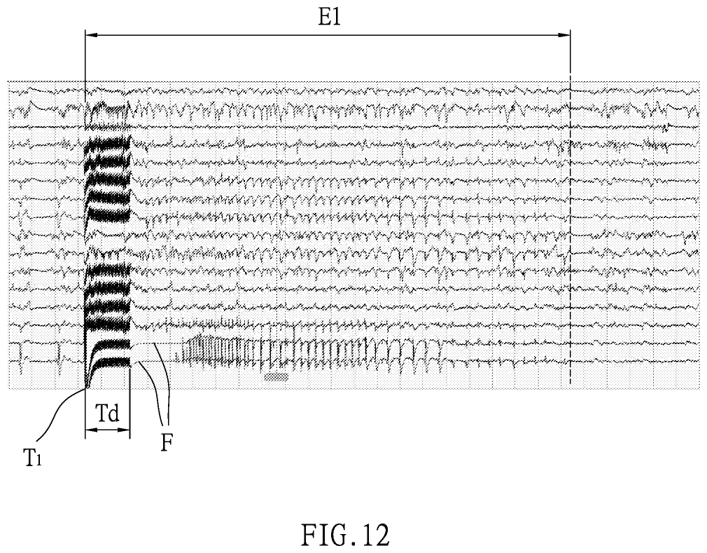

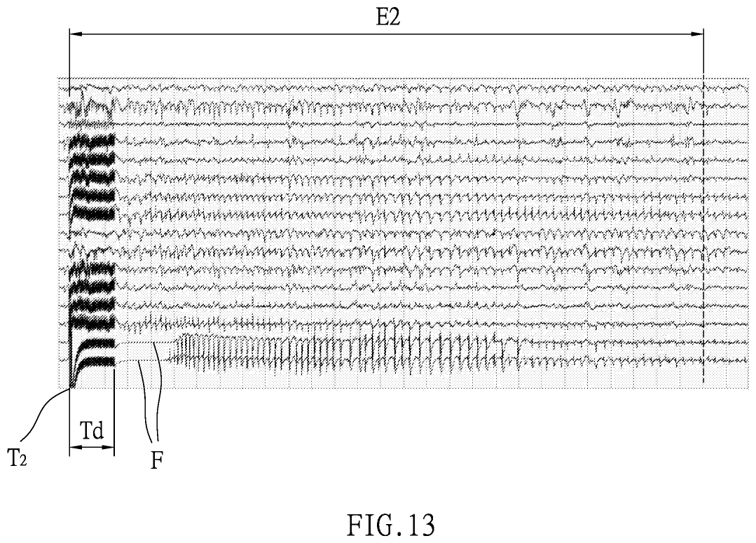

With regard to the (1) preparing step 11, it is needed to prepare a processing device 20 and a live animal 90 (such as pig, cow, or goat; preferably pig). The processing device 20 (as shown in FIGS. 8, 9 and 10) includes several electrodes 91C, several connecting wires 21, a controller 22 and a brainwave signal recorder 23. The electrodes 91C are connected with the controller 22 via these connecting wires 21. The brainwave signal recorder 23 can record brainwave signals (as exhibited in FIGS. 12 and 13) detected by these electrodes 91C. The controller 22 can apply electric stimulation to at least two electrodes 91C via the connecting wires 21. The live animal 90 (as illustrated in FIG. 7) has a brain portion 91. Furthermore, the brain portion 91 contains a cerebral cortex 911.

About the (2) anesthetizing step 12, as shown in FIG. 4, by using the traditional anesthetizing technique (via a breathing tube into its mouth with proper anesthetic gas), one operator can anesthetize the live animal 90 and monitor its biological information. After anesthetizing process is completed, a period of time lasts that is defined as an operation time.

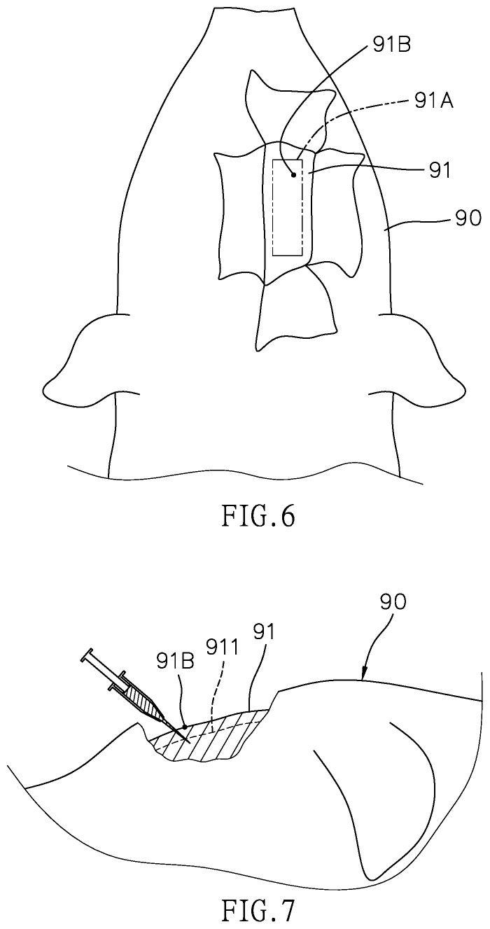

Concerning the (3) electrodes installing step 13, a head of the live animal 90 is opened for allowing the electrodes to be installed (or placed) on or in the brain portion 91 (as shown in FIG. 5). A working zone 91A (as shown in FIG. 6) is defined as a zone where the electrodes 91C installed on or in the brain portion 91. The working zone 91A includes a target position 91B (such as a specific point for electric stimulation).

About the (4) focal onset seizures inducing step 14, it includes the following processing steps:

[4a] at the beginning of the operation time, a first chemical liquid is injected beneath the cerebral cortex 911, as illustrated in FIG. 7; and the first chemical liquid can be Penicillin with the injection volume of 5000 IU.

[4b] during the operation time, a first chemical liquid is topped (or gently poured) on an outer surface of the cerebral cortex 911; and the first chemical liquid can be Penicillin with the volume of 10000 IU. For example, during a 4-hour operation time, the first chemical liquid is topped (or gently poured) four times (at the beginning and every hour thereafter).

[4c] at a first time T1 after the beginning of the operation time, activating a first electric stimulation so that the controller 22 is able to apply a first electric stimulation to at least two electrodes 91C via the connecting wires 21. The at least two electrodes 91C are near the target position 91B. After the first electric stimulation is applied, a first focal onset seizure E1 (as shown in FIG. 12) is generated on the live animal 90. Referring to FIG. 12 (or 13), there are 16 brainwave signals recorded by all these 16 electrodes (the top most wave is the output to the electrode labeled No. 1; the second top wave is the output of the electrode labeled No. 2, and so on) wherein the X axis means time, and the Y axis means voltage.

[4d] at a second time T2 after the beginning of the operation time, activating a second electric stimulation so that the controller 22 is able to apply a second electric stimulation to at least two electrodes 91C via the connecting wires 21. The at least two electrodes 91C are near the target position 91B. After the second electric stimulation is applied, a second focal onset seizure E2 (as shown in FIG. 13) is generated on the live animal 90.

Regarding the electrodes 91C, they can be positioned in a 2-dimensional arrangement and disposed on a thin film. In this preferred embodiment, it is 8×2 arrangement (or it can be altered to other type, like 4×5, 6×8, etc.). Please refer to FIG. 11 (corresponding to FIG. 10). There are 16 electrodes 91C. The lower left electrodes 91C is labeled as number 1. The upper left electrodes 91C is labeled as number 8. The lower right electrodes 91C is labeled as number 9. The upper right electrodes 91C is labeled as number 16. They are arranged sequentially. A first gap W in the horizontal direction can be 1 cm. A second gap G in the vertical direction can be 0.5 cm.

Furthermore, in one preferred embodiment, the operation time of an animal experiment is 4 fours. During the first 20 to 30 minutes, a slight brainwave fluctuation occurs. But it still does not reach the level of epilepsy. At the time of one hour and 49 minutes, the controller 22 activates a first electric stimulation to at least two electrodes 91C (the electrodes labeled 15 and labeled 16; near the target position 91B) via the connecting wires 21. The two electrodes 91C are near the target position 91B. After the first electric stimulation is applied, a first focal onset seizure (labeled E1 time range, lasting roughly 21 seconds) is generated on the live animal 90. The brainwave signals of all these 16 electrodes 91C can be seen in FIG. 12. The detailed condition of the first electric stimulation at the first time T1 is described as follows. Its lasting time Td is 2 seconds. Its current is 1-2 mA. Its pulse width is 500 ms, and its frequency is 50 Hz.

After which, there is no any epilepsy. At the time of one hour and 52 minutes, the controller 22 activates a second electric stimulation to two electrodes 91C (the electrodes labeled 15 and labeled 16; near the target position 91B) via the connecting wires 21. The two electrodes 91C are near the target position 91B. After the second electric stimulation is applied, a second focal onset seizure (labeled E2 time range, lasting roughly 27 seconds) is generated on the live animal 90. The brainwave signals of all these 16 electrodes 91C can be seen in FIG. 13. The detailed condition of the second electric stimulation at the first time T2 is described as follows. Its lasting time Td is 2 seconds. Its current is 1-2 mA. Its pulse width is 500 ms, and its frequency being 50 Hz.

Of course, the position, range, and/or condition for applying electric stimulation can be modified if needed. In addition, during the line animal is anesthetized, proper muscle relaxant can be injected as well.

Besides, please see the label F in FIGS. 12 and 13. Label F means that the fluctuation of the brainwave is too huge. The detected current is too large so that it exceeds the range that can be recoded. Therefore, it becomes a flat line.

Also, after a period of time, a third electric stimulation or further electric stimulation can be activated, if desired. As a result, a third or further focal onset seizure are generated accordingly. That is, this invention can create a predictable epilepsy. It is extremely convenient for the searcher to conduct epilepsy related animal model or experiment.

The advantages and functions of this invention can be summarized as follows.

[1] It can generate focal onset seizures. In this invention, the penicillin is injected before the first electric stimulation is activated. It is to establish as an environment that the focal onset seizures could be induced easier. Hence, once the electric stimulation is applied, the focal onset seizures will be generated and will last about 20 to 30 seconds averagely. Therefore, such focal onset seizures (or epilepsy) is predictable. It is a great breakthrough in the field of animal model for epilepsy.

[2] It is easy to change the electric stimulation position, numbers and type. In this invention, there are many electrodes. Not only the electrodes can record the brainwaves, but also certain current can be added via some of the electrodes. Therefore, it is easy to apply different types or modes of electric stimulations for the animal model test for epilepsy.

[3] The survival rate of a larger-sized animal after anesthesia is higher. About the traditional animal model test, the anesthetized period means the acute model. And, the period after anesthesia means the chronic model. Because this invention uses the larger-sized animal (such as pig) as the testing animal, its volume of its brain is larger for withstanding higher electric stimulation. Also, the damage of tissue is less. The survival rate becomes relatively higher. Therefore, it is suitable to continue to proceed further test in the period of the chronic model.

What is claimed is:

1. A method for producing an animal model for generating a stereotype of focal onset seizures comprising the steps of:
(1) preparing step: preparing a processing device and a live animal; said processing device including a plurality of electrodes, a plurality of connecting wires, a controller and a brainwave signal recorder; said electrodes being connected with said controller via said connecting wires, said brainwave signal recorder being able to record brainwave signals detected by said electrodes; said controller being able to apply electric stimulation to at least two electrodes via said connecting wires, said live animal having a brain portion, said brain portion containing a cerebral cortex;
(2) anesthetizing step: anesthetizing said live animal and monitoring its biological information; after anesthetizing process being completed, a period of time being defined as an operation time;
(3) electrodes installing step: a head of said live animal being opened for allowing said electrodes being installed on or in said brain portion; a working zone being defined as a zone where said electrodes installed on or in said brain portion; said working zone including a target position; and
(4) focal onset seizures inducing step: it including the following processing steps:
[4a] at the beginning of said operation time, a first chemical liquid being injected beneath said cerebral cortex;
[4b] during said operation time, a first chemical liquid being topped on an outer surface of said cerebral cortex;
[4c] at a first time after the beginning of said operation time, activating a first electric stimulation so that said controller being able to apply a first electric stimulation to at least two electrodes via said connecting wires, said at least two electrodes being near said a target position; after said first electric stimulation being applied, a first focal onset seizure being generated on said live animal; and
[4d] at a second time after the beginning of said operation time, activating a second electric stimulation so that said controller being able to apply a second electric stimulation to at least two electrodes via said connecting wires, said at least two electrodes being near said target position; said second electric stimulation causing second focal onset seizure generating of said live animal.

2. The method for producing an animal model for generating a stereotype of focal onset seizures as defined in claim 1, wherein said live animal being selected from a group consisting of pig, cow and goat; and said first chemical liquid being Penicillin.

3. The method for producing an animal model for generating a stereotype of focal onset seizures as defined in claim 1, wherein both said first and second electric stimulations being defined as: lasting time being 2 seconds, current being 1-2 mA, pulse width being 500 ms, and frequency being 50 Hz.
